(19) United States
(12) Patent Application Publication
(10) Pub. No.: US 2009/0264939 A9
(43) Pub. Date: Oct. 22, 2009

(54) INSTRUMENT SET AND METHOD FOR PERFORMING SPINAL NUCLECTOMY

(76) Inventors: Erik O. Martz, Savage, MN (US); Jean-Charles LeHuec, Bordeaux (FR); Michael Ahrens, Neustadt I.H. (DE)

Correspondence Address: STOEL RIVES LLP - SLC, 201 SOUTH MAIN STREET, SUITE 1100, ONE UTAH CENTER, SALT LAKE CITY, UT, 84111, US

(21) Appl. No.: 11/862,633

(22) Filed: Sep. 27, 2007

Related U.S. Application Data
(63) Continuation-in-part of application No. 11/304,053, filed on Dec. 15, 2005.
(60) Provisional application No. 60/636,777, filed on Dec. 16, 2004.

(51) Int. Cl. A61B 17/00; A61F 2/44; A61F 5/00; A61B 19/00; A61F 11/00

(52) U.S. Cl. 606/86 R; 623/17.16; 128/898; 606/79; 606/108; 623/17.16

(57) ABSTRACT

A nuclectomy method for creating a nuclear cavity in an annulus located in an intervertebral disc space and for preparing the nuclear cavity to receive an intervertebral prosthesis. The method involves identifying a plurality of regions in at least a portion of the nucleus. A sequence for removing the regions is also determined. At least one annulotomy is formed in the annulus along an annular axis to provide access to the nucleus. A guide system is positioned relative to the annulotomy. The guide system is configured to limit motion of at least one surgical tool relative to the guide system. A portion of the nucleus is removed from a first region using the surgical tool. At least one of the guide system and the surgical tool are configured to remove a portion of the nucleus from a second region. A portion of the nucleus is removed from a second region using the surgical tool.

### FIELD OF THE INVENTION

The present invention relates to a method and system for performing a spinal nuclectomy to create a nuclear cavity in an annulus located in an intervertebral disc space, and to prepare the nuclear cavity to receive an intervertebral prosthesis.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulpous, and annulus fibrosus. The central portion, the nucleus pulpous or nucleus is relatively soft and gelatinous; being composed of about 70 to 90% water. The nucleus pulpous has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to a number of injuries. Disc herniation occurs when the nucleus begins to extrude through an opening in the annulus, often to the extent that the herniated material impinges on nerve roots in the spine or spinal cord. The posterior and posterio-lateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine.

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization, such as for example U.S. Pat. No. 5,425,772 (Brantigan) and U.S. Pat. No. 4,834,757 (Brantigan).

Apparatuses and/or methods intended for use in disc repair have also been described but none appear to have been further developed, and certainly not to the point of commercialization. See, for instance, French Patent Appl. No. FR 2 639 823 (Garcia) and U.S. Pat. No. 6,187,048 (Milner et al.).

Prosthetic implants formed of biomaterials that can be delivered and cured in situ, using minimally invasive techniques to form a prosthetic nucleus within an intervertebral disc have been described in U.S. Pat. No. 5,556,429 (Felt); U.S. Pat. No. 5,888,220 (Felt et al.); U.S. Pat. No. 7,001,431 (Bao et al.); and U.S. Pat. No. 7,077,865 (Bao et al.), the disclosures of which are incorporated herein by reference. Related methods are disclosed in U.S. Pat. No. 6,224,630 (Bao et al.), entitled “Implantable Tissue Repair Device” and U.S. Pat. No. 6,079,868 (Rydell), entitled “Static Mixer” the disclosures of which are incorporated herein by reference.

The methods of these references include, for example, the steps of inserting a mold apparatus (which in a preferred embodiment is described as a “mold”) through an opening within the annulus, and filling the mold to the point that the mold material expands with a flowable biomaterial that is adapted to cure in situ and provide a permanent disc replacement.

Nucleus replacement requires a simple and reliable method of removing the anatomical nucleus. Care must be taken to avoid damage to the annulus and the bony end plates of the adjacent vertebrae. The nuclear cavity is preferably symmetrical and centered along the axis of the spine. For many patients,

### BRIEF SUMMARY OF THE INVENTION

The present invention relates to a method and apparatus for performing a spinal nuclectomy to remove at least a portion of a nucleus from an a disc space to create a nuclear cavity in an intervertebral disc space, and to prepare the nuclear cavity to receive an intervertebral prosthesis. Various guide systems are disclosed to direct and limit the motion of the surgical tools in the instrument set during the procedure. The guide systems can provide visual, tactile and/or auditory signals to assist the surgeon.

The guide system can be part of a surgical tool or a separate structure. The guide system can optionally be attached to the surgical table, a catheter holder used to implant a spinal prosthesis, to the patient, or a variety of other structures in the operating room.

In one embodiment, the nuclectomy method includes removing at least a portion of a nucleus from an annulus to create a nuclear cavity in an intervertebral disc space and preparing the nuclear cavity to receive an intervertebral prosthesis. A plurality of regions in at least a portion of the nucleus and a sequence for removing the regions is identified. At least one annulotomy is formed in the annulus along an annular axis to provide access to the nucleus. A guide system is positioned relative to the annulotomy. The guide system is configured to limit motion of at least one surgical tool relative to the guide system. A portion of the nucleus is removed from a first region using the surgical tool. At least one of the guide system and the surgical tool are configured to remove a portion of the nucleus from a second region. A portion of the nucleus is removed from a second region using the surgical tool.

The guide system can be positioned inside or outside the intervertebral disc. The same or different surgical tools can be used to remove the nucleus from the first and second regions. The guide system can limit movement of the surgical tool relative to the guide system to one or two degrees of freedom.

In one embodiment, the geometry of the intervertebral disc space is evaluated prior to surgery using imaging techniques, such as for example, an x-ray, MRI, CAT-scan, or ultrasound. By knowing the geometry of the nucleus and/or the annulus, and the trajectory of the surgical approach into the nucleus, the present guide system and the surgical tools can be configured to perform each step of the nuclectomy procedure.

The present instrument set is preferably configured and sequenced before the surgery based on the geometry of the intervertebral disc space of the particular patient. Alternatively, the surgeon has the option to make adjustments to the guide system and/or instrument set during the procedure.

In one embodiment, a standard instrument set and guide system configuration and sequence is prepared for a particular entry path into the nucleus. The surgeon has the option to make adjustments during the procedure. The method and apparatus disclosed herein can be used for a single annulotomy procedures or multi-annulotomy procedures.

The surgeon preferably performs the nuclectomy using the pre-configured and pre-sequenced guide system and instrument set. The systematic approach to nuclectomy disclosed herein increases the likelihood that all of the targeted nucleus material will be removed, the nuclear cavity will be centered within the disc space, and/or the nuclear cavity will be symmetrical relative to the midline of the spine.

The step of evaluating the geometry of the nuclear cavity also provides an indication of the total volume. In one embodiment, an evaluation mold is positioned in the nuclear cavity and a fluid is delivered to the evaluation mold so that the mold substantially fills the nuclear cavity. The evaluation mold can be used to estimate the quantity of nucleus material removed at any point in the nuclectomy procedure, as well as the position and shape of the nuclectomy cavity. Evaluating the quantity of nucleus material removed, as well as the position and shape of the resultant cavity, can be a primary or secondary method of determining whether the nuclectomy is completed.

In one embodiment, the method includes forming first and second annulotomies in the annulus. A portion of the nucleus is removed through the first annulotomy using at least a first surgical tool and a portion of the nucleus is removed through the second annulotomy using at least a second surgical tool.

As used herein the following words and terms shall have the meanings ascribed below:

“biomaterial” will generally refer to a material that is capable of being introduced to the site of a joint and cured to provide desired physical-chemical properties in vivo. In one embodiment the term will refer to a material that is capable of being introduced to a site within the body using minimally invasive mechanism, and cured or otherwise modified in order to cause it to be retained in a desired position and configuration. Generally such biomaterials are flowable in their uncured form, meaning they are of sufficient viscosity to allow their delivery through a delivery tube of on the order of about 1 mm to about 10 mm inner diameter, and preferably of about 2 mm to about 5 mm inner diameter. Such biomaterials are also curable, meaning that they can be cured or otherwise modified, in situ, at the tissue site, in order to undergo a phase or chemical change sufficient to retain a desired position and configuration;

“cure” and inflections thereof, will generally refer to any chemical transformation (e.g., reacting or cross-linking), physical transformation (e.g., hardening or setting), and/or mechanical transformation (e.g., drying or evaporating) that allows the biomaterial to change or progress from a first physical state or form (generally liquid or flowable) that allows it to be delivered to the site, into a more permanent second physical state or form (generally solid) for final use in vivo. When used with regard to the method of the invention, for instance, “curable” can refer to uncured biomaterial, having the potential to be cured in vivo (as by catalysis or the application of a suitable energy source), as well as to the biomaterial in the process of curing. As further described herein, in selected embodiments the cure of a biomaterial can generally be considered to include three stages, including (a) the onset of gelation, (b) a period in which gelation occurs and the biomaterial becomes sufficiently tack-free to permit shaping, and (c) complete cure to the point where the biomaterial has been finally shaped for its intended use.

“minimally invasive mechanism” refers to a surgical mechanism, such as microsurgical, percutaneous, or endoscopic or arthroscopic surgical mechanism, that can be accomplished with minimal disruption to the annular wall (e.g., incisions of less than about 4 cm and preferably less than about 2 cm). In some embodiments, minimally invasive mechanisms also refers to minimal disruption of the pertinent musculature, for instance, without the need for open access to the tissue injury site or through minimal skin incisions. Such surgical mechanism are typically accomplished by the use of visualization such as fiberoptic or microscopic visualization, and provide a post-operative recovery time that is substantially less than the recovery time that accompanies the corresponding open surgical approach.

“mold” will generally refer to the portion or portions of an apparatus of the invention used to receive, constrain, shape and/or retain a flowable biomaterial in the course of delivering and curing the biomaterial in situ. A mold may include or rely upon natural tissues (such as the annular shell of an intervertebral disc) for at least a portion of its structure, conformation or function. The mold, in turn, is responsible, at least in part, for determining the position and final dimensions of the cured prosthetic implant. As such, its dimensions and other physical characteristics can be predetermined to provide an optimal combination of such properties as the ability to be delivered to a site using minimally invasive mechanism, filled with biomaterial, prevent moisture contact, and optionally, then remain in place as or at the interface between cured biomaterial and natural tissue. In one embodiment the mold material can itself become integral to the body of the cured biomaterial. The mold can be elastic or inelastic, permanent or bio-reabsorbable, porous or non-porous.

### BRIEF DESCRIPTION OF THE SEVERAL VIEWS OF THE DRAWING

FIG. 1 is an exemplary prior art catheter and mold.

FIG. 2 is a schematic illustration of various entry paths for use in accordance with the present invention.

FIG. 3 is a side sectional view of a guide system in accordance with an embodiment of the present invention.

FIG. 4 is a sectional view of the guide system of FIG. 3 in a horizontal configuration in accordance with an embodiment of the present invention.

FIG. 5 is a side view of a surgical tool with a guide system in accordance with an embodiment of the present invention.

FIG. 6 is a side view of an alternate surgical tool with a guide system in accordance with an embodiment of the present invention.

FIG. 7 is a top view of the surgical tool of FIG. 6.

FIG. 8 is a side view of an alternate guide system in accordance with an embodiment of the present invention.

FIG. 9 is a perspective view of an adaptor for use in a guide system in accordance with an embodiment of the present invention.

FIG. 10 is a perspective view of an alternate adaptor for use in a guide system in accordance with an embodiment of the present invention.

FIG. 11 is a side view of an alternate surgical tool with a guide system in accordance with an embodiment of the present invention.

FIG. 12 is a top view of the surgical tool of FIG. 12.

FIG. 13 is a side view of an alternate surgical tool with a guide system in accordance with an embodiment of the present invention.

FIG. 14A is an end view of the guide system of FIG. 13.

FIG. 14B-14D are a side sectional views of alternate guide systems in accordance with an embodiment of the present invention.

FIG. 15 is a side sectional view of the surgical tool and guide system of FIG. 13 engaged with a patient in accordance with an embodiment of the present invention.

FIG. 16 is a side sectional view of an alternate surgical tool with a guide system in accordance with an embodiment of the present invention.

FIG. 17 is a side sectional view of the surgical tool of FIG. 16 in an extended configuration.

FIGS. 18 through 23 are a horizontal sectional views of a method and guide system performing a nuclectomy in accordance with an embodiment of the present invention.

FIGS. 24 and 25 are a horizontal sectional views of a method and guide system performing a multi-portal nuclectomy in accordance with an embodiment of the present invention.

FIG. 26 illustrates an alternate guide system in accordance with an embodiment of the present invention.

### DETAILED DESCRIPTION OF THE INVENTION

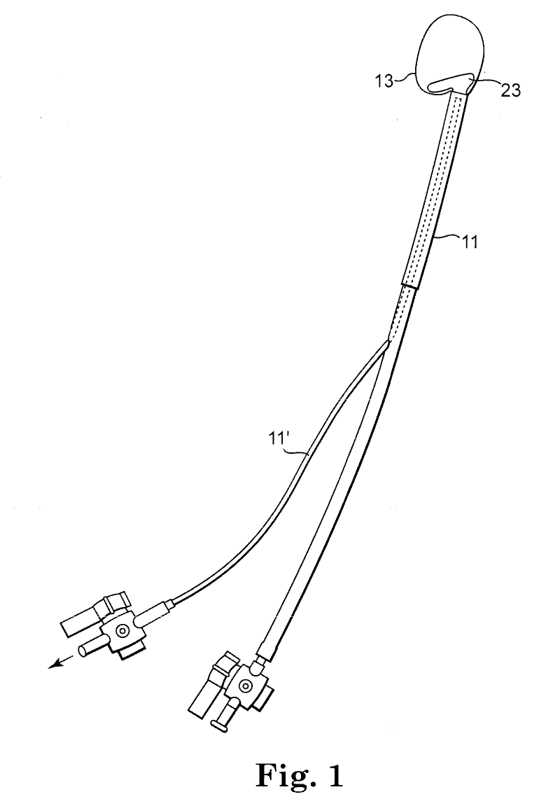

The present nuclectomy method is the preferred precursor procedure to implanting certain intervertebral prostheses. FIG. 1 illustrates an exemplary prior art catheter 11 with mold or balloon 13 located on the distal end for an in situ curable prosthetic implant. In the illustrated embodiment, biomaterial 23 is delivered to the mold 13 through the catheter 11. Secondary tube 11′ evacuates air from the mold 13 before, during and/or after the biomaterial 23 is delivered. The secondary tube 11′ can either be inside or outside the catheter 11. A flowable biomaterial 23 is delivered through a catheter 11 into the mold located in the annulus. The delivered biomaterial 23 is allowed to cure a sufficient amount to permit the catheters 11 and 11′ to be removed. Various implant procedures, implant molds, and biomaterials related to intervertebral disc replacement suitable for use with the present system and apparatus are disclosed in U.S. Pat. No. 5,556,429 (Felt); U.S. Pat. No. 6,306,177 (Felt, et al.); U.S. Pat. No. 6,248,131 (Felt, et al.); U.S. Pat. No. 5,795,353 (Felt); U.S. Pat. No. 6,079,868 (Rydell); U.S. Pat. No. 6,443,988 (Felt, et al.); U.S. Pat. No. 6,140,452 (Felt, et al.); U.S. Pat. No. 5,888,220 (Felt, et al.); U.S. Pat. No. 6,224,630 (Bao, et al.); U.S. Pat. No. 7,001,431 (Bao et al.); and U.S. Pat. No. 7,077,865 (Bao et al.); and U.S. Pat. Publication No. 2006/0253199 entitled Lordosis Creating Nucleus Replacement Method and Apparatus, all of which are hereby incorporated by reference.

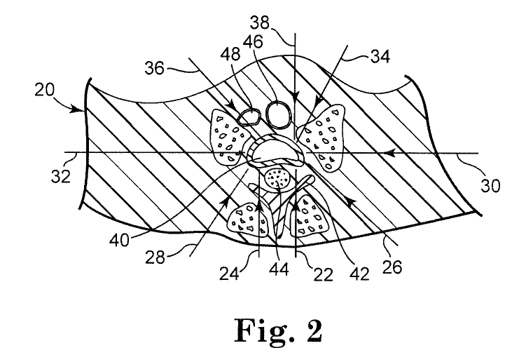

FIG. 2 is a cross-sectional view of a human body 20 showing exemplary entry paths 22 through 38 to the intervertebral disc 40 suitable for use in performing the method of the present invention. The posterior paths 22, 24 extend either between superior and inferior transverse processes 42, or between the laminae (interlaminar path) on either side of the spinal cord 44. The posterolateral paths 26, 28 are also on opposite sides of the spinal cord 44 but at an angle of about 35-45 degrees relative to horizontal relative to the posterior paths 22, 24. The lateral paths 30, 32 extend through the side of the body. The anterior path 38 and anterolateral path 34 extend past the aorta iliac artery 46, while the anterolateral path 36 is offset from the inferior vena cava, iliac veins 48.

The surgeon selects the entry path 22-38 depending on the disc level being operated on, and the patient anatomy. Generally, the aorta and vena cava split at the L4 vertebral body. At L5S1 the approach is typically a midline anterior approach. At L4/5 the approach may be either midline anterior or anterolateral, depending on the patient anatomy and how easy it is to retract the vessels. In some usages, the anterior approach is deemed a midline approach and the anterolateral approach is deemed an angled approach offset from the midline anterior approach.

The present method and apparatus use one or more of the access paths 22 through 38. While certain of the access paths 22 through 38 may be preferred depending on a number of factors, such as the nature of the procedure, any of the access paths can be used with the present invention.

In one embodiment, guide systems are positioned along two or more of the access paths 22 through 38 to facilitate preparation of the intervertebral disc 40. Preparation includes, for example, formation of two or more annulotomies through the annular wall, removal of some or all of the nucleus pulposus to form a nuclear cavity, imaging of the annulus and/or the nuclear cavity, and positioning of a multi-lumen mold in the nuclear cavity. The multi-portal approach is particularly suited for use with the multi-lumen molds disclosed in U.S. Pat. Publication No. 2006/0253198, entitled Multi-Lumen Mold For Intervertebral Prosthesis And Method Of Using Same, previously incorporated by reference. Guide systems according to various embodiments are suitable for accessing the annulus from any of the available access directions, including posterior, posterior lateral, lateral, anterior, or anterolateral.

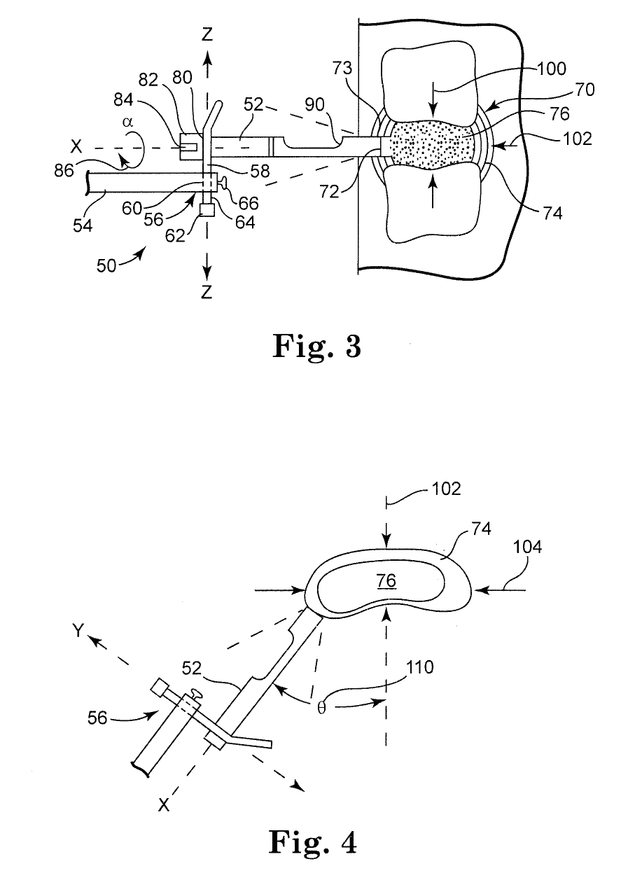

FIG. 3 is a side sectional view of a guide system 50 in accordance with an embodiment of the present invention. The guide system 50 can preferably control motion of surgical tools through six degrees of freedom. In the illustrated embodiment, the six degrees of freedom include the X axis, Y axis, and Z axis, as well as pitch (rotation around the Y axis), roll (rotation around the X axis) and yaw (rotation around the Z axis). While it is possible to construct a guide system in accordance with the present invention having less than six degrees of freedom, six are preferred. As used herein, the phrases “limit motion” and “limiting motion” refer to restricting displacement of a surgical tool relative to a guide system in at least one of the six degrees of freedom.

In the illustrated embodiment, the mounting fixture 54 is attached to a secondary holding device (not shown) that is preferably attached, directly or indirectly through additional components, to some fixed structure, such as an operating table. In another embodiment, the secondary holding device can include a handle that is gripped by a member of the operating staff to hold the guide system 50 in the desired location. In yet another alternate embodiment, the secondary holding device is attached, directly or indirectly through additional components, to the patient, such as for example, using a retractor, Steinmann pins, a harness fitted to the patient, or a variety of other devices. As used herein, “secondary holding device” refers to a mechanism that can be, directly or indirectly through additional components, releasably attached to the patient, releasably attached to an external structure, gripped by the surgical staff, or any combination thereof.

In the illustrated embodiment, guide 52 is hollow to provide access to the intervertebral disc space 70. In alternate embodiments, the guide 52 can be a rail, a shaft, or a variety of other structures. During the procedure, an annulotomy 73 is made in the annulus 74 to provide access to the nucleus 76.

Distal end 72 of the guide 52 preferably contacts annulus 74. In the illustrated embodiment, the distal end 72 extends into the annulotomy 73. It is also possible for the distal end 72 to contact the nucleus 76.

The guide 52 is attached to mounting fixture 54 by slide mechanism 56. Slide mechanism 56 includes an elongated portion 58 that slides in a channel 60 in the mounting fixture 54. Adjustable stop 62 is provided on distal end 64 of the elongated member 58 to limit the range of motion of the guide 52 around the Y axis (pitch). Set screw 66 is provided to secure the guide 52 at a particular position along the length of the elongated member 58. The slide mechanism 56 permits the pitch of the guide 52 to be controlled before and/or during the surgical procedure. An alternate structure is disclosed in U.S. Patent Publication No. 2006/0265076 entitled Catheter Holder for Spinal Implant, which is hereby incorporated by reference.

The guide 52 can also be used as an access port for performing other steps in the procedure. For example, the proximal end 72 can be used for evaluating the nuclectomy or the annulus 74; imaging the nucleus 76; implanting the mold 13; delivering the biomaterial; and/or cutting the catheter 11 as close to the neck of the mold 13 as possible. Disclosure related to evaluating the nuclectomy or the annulus is found in U.S. Pat. Publication No. 2005/0209602, entitled “Multi-Stage Biomaterial Injection System for Spinal Implants, which is incorporated by reference.

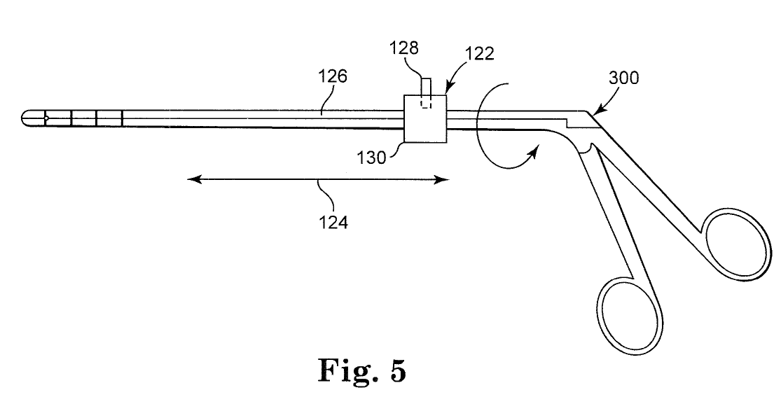

In the illustrated embodiment, proximal end 80 of the guide 52 includes adaptor 82. The adaptor 82 includes a slot 84 adapted to engage with a stop on a surgical tool (see e.g., FIG. 5). In this embodiment, the slot 84 controls both the rotation 86 of the surgical tool around the X axis (roll) and the depth of penetration into the nucleus 76 along the X axis. The adaptor 82 is preferably releasably attached to proximal end 80 of the guide 52 so as to permit a variety of different adaptors to be used during the nuclectomy. In another embodiment, the adaptor 82 can be indexed to particular locations around the X axis to permit certain portions of the nucleus 76 to be removed.

In another embodiment, slot 90 is provided in the guide 52 near the distal end 72. In this embodiment, a feature on the surgical tools can be constrained by engagement with the slot 90, as will be discussed in detail below.

FIG. 4 is a horizontal sectional view of the intervertebral disc space 70 discussed above. Slide mechanism 56 has been reconfigured to permit horizontal motion of the guide 52 around the Z axis (yaw).

In one embodiment, the geometry of the intervertebral space 70 is evaluated prior to the surgical procedure using imaging techniques. The imaging techniques preferably identify the height 100, depth 102, and width 104 of the nucleus 76. By knowing the geometry of the nucleus 76 and the trajectory through the annulus 74, it is possible to configure the guide system 50 and surgical tools for each step of the nuclectomy procedure. In the embodiment illustrated in FIG. 4, offset angle 110 defines the trajectory along the X axis through the annulus 74. Consequently, it is possible to identify and sequence a plurality of guide system configurations, adaptors, and surgical instruments prior to beginning the nuclectomy procedure. This method permits the surgeon to customize the procedure for each patient, while maintaining efficiency.

FIG. 5 is a side sectional view of a straight rongeur 120 including a guide system 122 in accordance with an embodiment of the present invention. In the illustrated embodiment, the guide system 122 is a cylindrical member 130 that slides along length 124 of shaft 126. A fastener 128, such as for example a set screw, pin, knob, protrusion, or other structure, can be used to secure the guide system 122 to a fixed location along the length of the shaft 126.

In one embodiment, the set screw 128 acts as a stop that engages with slot 84 on the adaptor 82 illustrated in FIG. 3. The set screw 128 thereby limits the depth of penetration along the X axis and the rotation 86 around the X axis (roll). By adjusting the location of the guide system 122 along the length of the shaft 126, the surgeon can change the depth of penetration into the nucleus 76 along the X axis. While this embodiment limits maximum penetration, minimum penetration is at the discretion of the surgeon.

In an alternate embodiment, the set screw 128 is temporarily removed and the guide system 122 is slid further along the length of the shaft 126. The set screw 128 is then engaged with the guide system 122 so that it is positioned in the slot 90 on the guide 52 illustrated in FIG. 3. The slot 90 limits both the maximum and the minimum depth of penetration along the X axis. As with the slot 84, the slot 90 also limits the rotation around the X axis.

Alternatively, protrusion 122 is optionally a spring-loaded detent, that can be depressed into the cylindrical member 130 to allow it to enter the guide 52. Once the protrusion 122 reaches the slot 90, the spring forces the protrusion 122 up, which limits motion of the rongeur 120 within the slot 90.

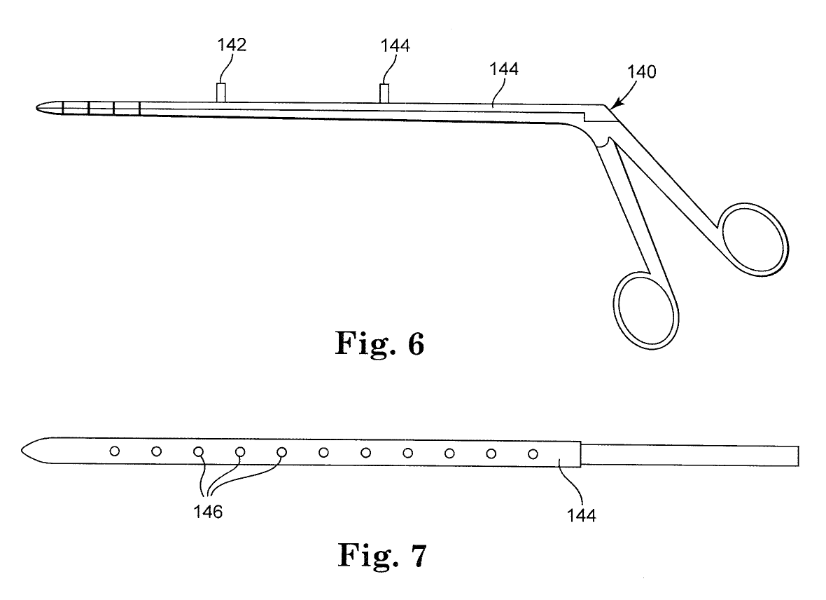

FIG. 6 is a side view of an alternate straight rongeur 140 in accordance with an embodiment of the present invention. In the embodiment of FIG. 6, one or more adjustable stops 142 are attached to the shaft 144 of the surgical tool 140. As illustrated in FIG. 7, top of the shaft 144 includes a plurality of holes 146 that are adapted to receive one or more adjustable stops 142. The embodiment of FIG. 6 and 7 is particularly well suited for use with the slot 90 in the guide 52 illustrated in FIG. 3. Since the length of the slot 90 is fixed, the surgeon can easily adjust the minimum and maximum depth of penetration by adjusting the location of one or more adjustable stops 142 in the threaded holes 146.

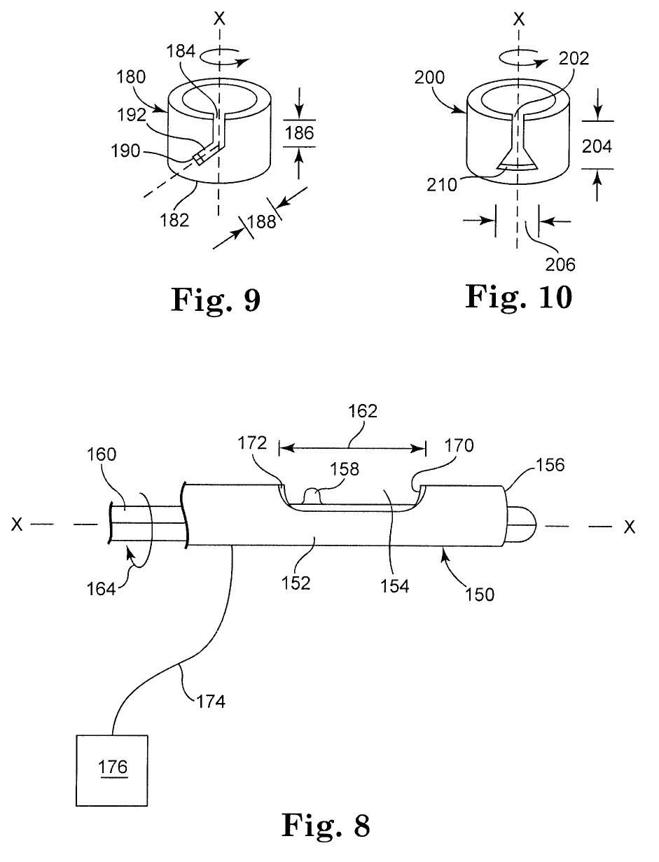

FIG. 8 is a side sectional view of an alternate guide system 150 in accordance with an embodiment of the present invention. In the illustrated embodiment, member 152 is a hollow tubular member with a slot 154 near distal end 156. Stop 158 on surgical tool 160 is positioned to traverse length 162 of the slot 154. The length of the slot 162 limits maximum and minimum penetration of the surgical tool 160 along the X axis. Since the length 162 of the slot is fixed, a second stop can optionally be attached to the surgical tool 160 to change the maximum and minimum penetration. The width of the slot limits the rotation 164 around the X axis and angulation relative to the X axis.

In one embodiment, distal end 156 is coupled to proximal end 80 of the guide 52 illustrated in FIG. 3. In an alternate embodiment, distal end 156 can be used free hand, or coupled to plate 260 positioned located on the patent (see e.g., FIG. 15).

In one embodiment, guide system 150 includes sensors 170, 172 at the distal and proximal ends of the slot 154. When the stop 158 engages one of the sensors 170, 172 a signal is sent via cable 174 to signal generator 176. The signal generator can provide auditory, visual, and/or tactile signals to the surgeon indicating the maximum and minimum penetration of the surgical tool 160.

FIG. 9 illustrates an alternate adaptor 180 for the guide systems in accordance with embodiments of the present invention. The adaptor 180 can be used free-hand or distal end 182 can optionally be attached to a support structure, such as for example, the proximal end 80 of the guide 52 illustrated in FIG. 3 or the plate 260 in FIG. 15.

The adaptor 180 includes a slot 184 that directs the surgical tool down the X axis to a particular depth 186. Once the depth 186 has been reached, the angled portion 192 permits an angular offset 188 of the surgical tool. Sensor 190 is optionally located at the distal end of the angular offset 188. By selecting the appropriate surgical tool, the adaptor 180 directs the surgeon to a particular location in the nucleus 76. The adaptor 180 can be indexed around the X axis to remove remote portions of the nucleus 76.

FIG. 10 illustrates an alternate adaptor 200 in accordance with an embodiment of the present invention. Slot 202 has a depth 204. Distal end of the slot 202 includes a width 206 that will permit rotation of the surgical tool around the X axis. The slot 202 of FIG. 10 constrains rotation and/or angulation of the surgical tool initially, but permits limited rotation and/or angulation when the full depth of penetration is achieved. Sensor 210 is optionally provided at distal end of the slot 202 to signal the surgeon that rotation and/or angulation is now permitted. In the illustrated embodiment, the width 206 of the slot 202 permits the surgical tool to be rotated approximately 15 degrees. The adaptor 200 can be indexed around the X axis to remove remote portions of the nucleus 76. Alternate adaptors can be provided that permit rotation at any depth up to a full 360 degrees.

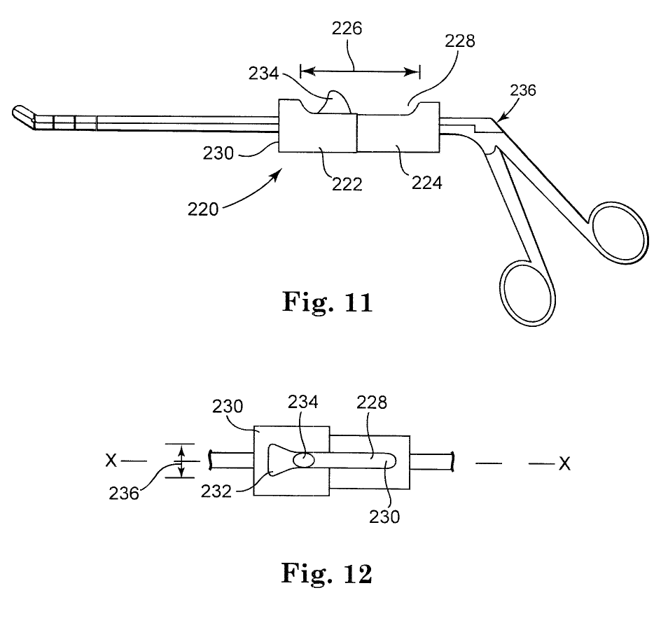

FIGS. 11 and 12 illustrate an alternate guide system 220 in accordance with an embodiment with the present invention. The guide system 220 includes a first portion 222 telescopically engaged with second portion 224. By adjusting the relative positions of the first and second portions 222, 224 the length 226 of the slot 228 can be adjusted. The guide system 220 can be used free-hand or distal end 230 of the guide system 220 can optionally be attached to a support structure, such as for example, the proximal end 80 of the guide 52 illustrated in FIG. 3 or the plate 260 in FIG. 15.

FIG. 12 illustrates a top view of the slot 228. The slot 228 includes a straight portion 230 and a flared portion 232. The stop 234 limits rotation and/or angulation of the surgical tool 236 around the X axis until the stop 234 reaches the flared portion 232. With 236 of the flared portion 232 determines the angular rotation permitted by the guide system 220.

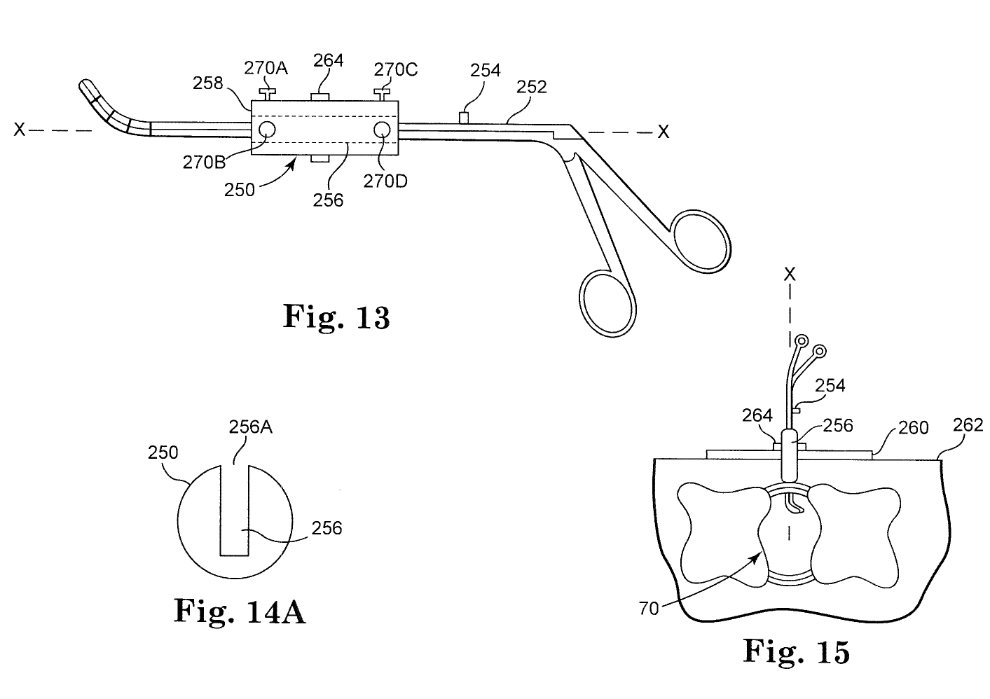

FIG. 13 is a side view of an alternate guide system 250 in accordance with another embodiment of the present invention. Curved rongeur 252 includes one or more stops 254 that limit movement relative to the guide system 250.

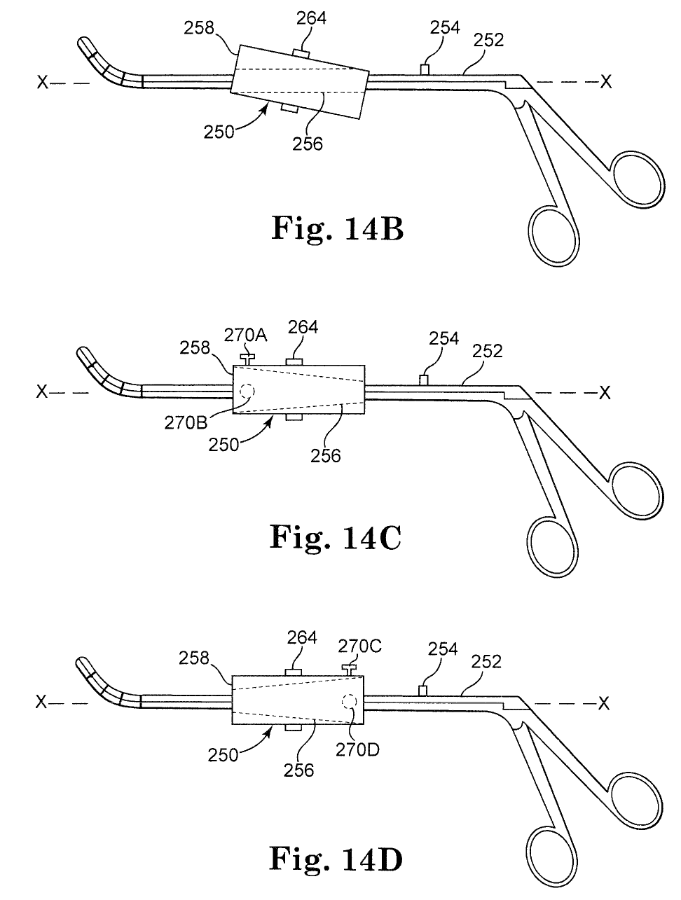

FIGS. 14A-14D illustrate exemplary embodiments of the guide system 250 with an opening 256 that directs or controls movement of the curved rongeur 252. FIG. 14A is an end view of the guide system 250 with an opening 256 that limits rotation of the rongeur 252 around the X axis. In the embodiment of FIG. 14A, the guide system 250 is an open structure with entrance 256A to facilitate engagement with the surgical tool 252. FIG. 14B is a side sectional view of the guide system 250 with an angled opening 256. FIG. 14C is a side sectional view of the guide system 250 with an opening 256 that flares outward toward the distal end 258 to permit angulation. FIG. 14D is a side sectional view of the guide system 250 with an opening 256 that flares inward toward the distal end 258 to permit angulation.

In embodiments where the guide system 250 includes an opening 256 with a cross-sectional area greater than a cross-sectional area of the curved rongeur 252, one or more limits 270A-270D (referred to collectively as “270”), such as for example set screws, protrusions, pins, and the like, are optionally provided to limit movement of the curved rongeur 252 to a particular path or range of motion. For example, the set screws 270A and 270B in FIGS. 14C and 14D can be adjusted to limit angulation of the rongeur 252 relative to the guide system 250.

In one embodiment, distal end 258 of the guide system 250 can be coupled to proximal end 80 of the guide 52 illustrated in FIG. 3. In an alternate embodiment illustrated in FIG. 15, guide system 250 is coupled with plate 260 located on the surface of the patient 262. Stops 264 on the guide system 250 limit maximum penetration of the guide system 250 relative to the plate 260. The rotational position of the guide system 250 relative to the plate 260 is used to control and limit rotation around the X axis. Stop 254 on the curved rongeur 252 limits penetration of the surgical tool into the intervertebral disc space 70.

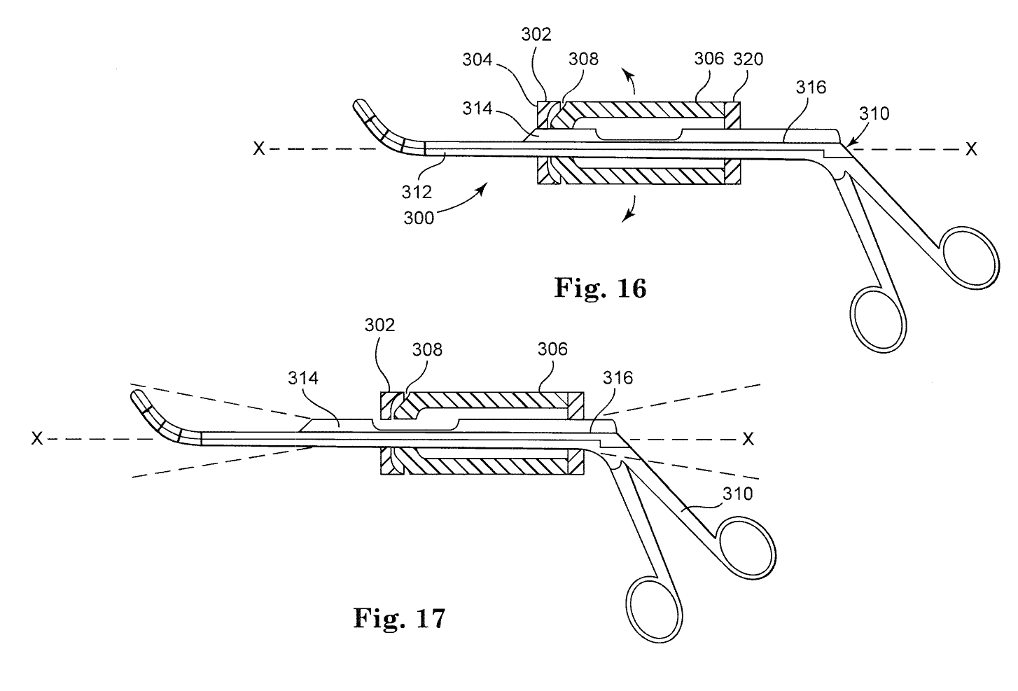

FIG. 16 illustrates an alternate guide system 300 in accordance with an embodiment of the present invention. The guide system 300 includes a fixed portion 302 with a distal end 304 that optionally can be attached to proximal end 80 of the guide 52 illustrated in FIG. 3. Articulating portion 306 couples with the fixed portion 302 at interface 308.

Surgical tool 310 is a curved rongeur in the illustrated embodiment. Shaft 312 of the curved rongeur 310 includes front ridge 314 and rear ridge 316. In the configuration illustrated in FIG. 16 the front ridge 314 engages with the fixed portion 302 and the articulating portion 306 at the interface 308, preventing articulation. The rear ridge 316 optionally couples with end cap 320 at the proximal end of the guide system 300. In the configuration illustrated in FIG. 16, the curved rongeur 310 can move along the X axis, but cannot move through pitch or yaw along the Z axis or Y axis.

As illustrated in FIG. 17, as the curved rongeur 310 is advanced along the X axis the front ridge 314 disengages from the interface 308, permitting the articulating portion 306 to move relative to the fixed portion 302. Depending on the configuration of the interface 308, the curved rongeur 310 can now move along the Y axis (yaw) and/or the Z axis (pitch).

By changing the length of the front ridge 314, the guide system can be configured to restrict motion to the X axis until the target depth is reached. Once the target depth is reached, the surgical tool 310 can be rotated in the Y direction and/or Z direction. In one embodiment, the amount of articulation is controlled by the configuration of the interface 308. In an alternate embodiment, the amount of articulation is controlled by the height of the front ridge 314. For example, a sloped or angled front ridge 314 would permit progressively more or less pitch and/or yaw movement of the surgical tool 310 relative to the fixed portion 302. The guide system 300 can optionally be configured to limit motion around the X axis.

The present method and apparatus are directed to an improved nuclectomy or total nucleus removal (TNR). Total nucleus removal refers to removal of substantially all of the nucleus from an intervertebral disc. In one embodiment, total nucleus removal is preferably removal of at least 70% of the nucleus, and more preferably at least 80% of the nucleus is removed, and most preferably at least 90% of the nucleus is removed from the intervertebral disc.

The TNR is the preferred precursor procedure for deploying a nucleus replacement prosthesis, such as for example an inflatable or expandable prosthesis, a fixed geometry prosthesis, delivering a curable biomaterial directly into the nuclear cavity, a self-expanding prosthesis, and the like. The present TNR methodology permits the nucleus replacement prosthesis to be accurately and symmetrically positioned within an intervertebral disc space.

In one embodiment, the nucleus is divided into a plurality of regions. A preferred sequence for removing the nucleus material from each of the regions is established. The regions are preferably arranged to take into consideration the three-dimensional nature of the nucleus material. Various sequences for performing a nuclectomy are disclosed in U.S. Pat. Publication No. 2006/0253199 entitled Nuclectomy Method and Apparatus, which is hereby incorporated by reference.

At least two different surgical instruments are typically used to remove the nucleus material from at least two of the regions. The surgical instruments are selected for optimum removal of the nucleus material from a given region. In some embodiments, reconfiguring the guide system permits a single surgical tool to be used to remove the nucleus material from two of the regions. In some embodiments, indicia are provided on the surgical tools to measure depth of penetration into the annulus.

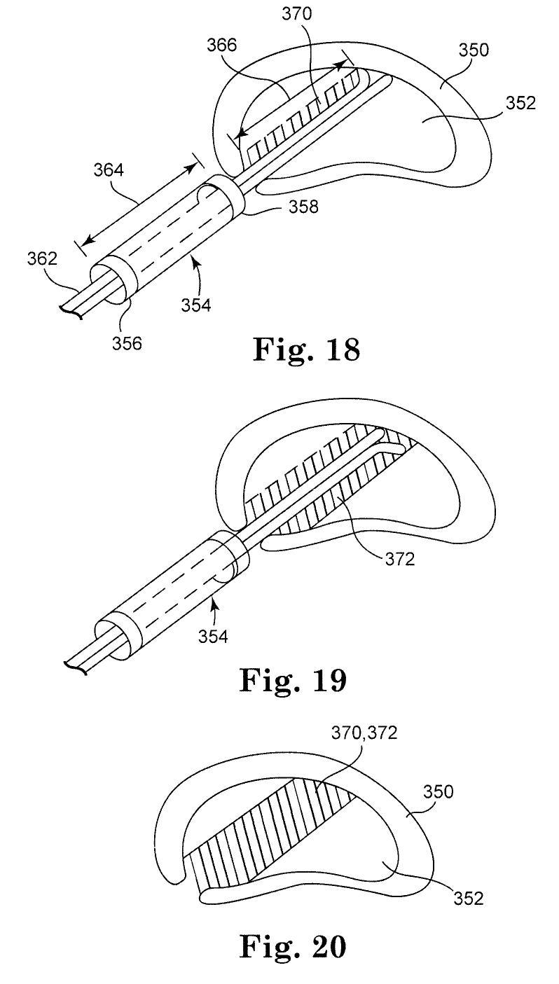

FIGS. 18-23 illustrate a nuclectomy performed using the method and instrument set in accordance with one embodiment of the present invention. The disc is preferably evaluated prior to surgery using conventional imaging techniques. The geometry of the disc is therefore known with some degree of certainty. The trajectory of the surgical approach for the nuclectomy is also predetermined. Based on this information, the guide system and surgical tools are configured to perform the nuclectomy before the procedure.

FIG. 18 illustrates guide system 354 orientated in a posterolateral entry configuration. End caps 356, 358 limit maximum and minimum movement of the stop 360 on the surgical tool 362. The depth of travel 364 permitted by the guide system 354 corresponds to the target depth of penetration 366 into the nucleus 352.

In the step illustrated in FIG. 18, straight rongeur 352 is used to remove nucleus material in region 370. As illustrated in FIG. 19, the guide system 354 is rotated 180 degrees so that the straight rongeur 362 can also be used to remove nucleus material from region 372. Due to the three-dimensional nature of the nucleus 76, the guide system 354 may optionally be rotated 180 degrees on 60 degree increments, removing more nuclear material at each step. FIG. 20 illustrates the annulus 350 with nucleus material 352 removed from regions 370, 372.

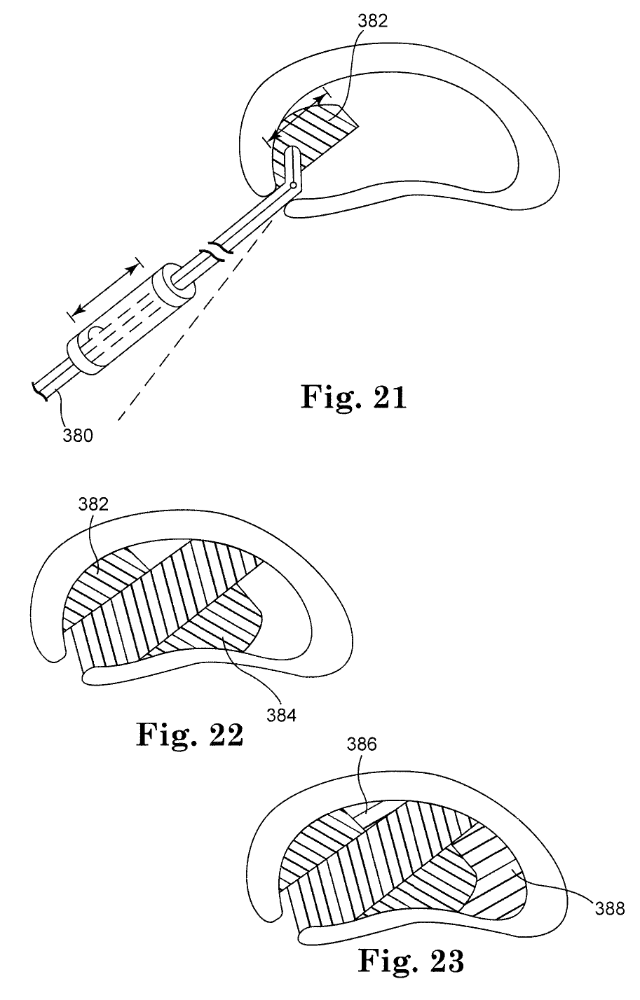

FIG. 21 illustrates the use of up angled rongeur 380 in the guide system 354 to remove nucleus material from region 382. The guide system 354 is then rotated 180 degrees to permit the same up angled rongeur 380 to remove nucleus material 352 from region 384 (see FIG. 22). Finally, curved rongeur (see FIG. 13) is used to remove nucleus material 352 from the regions 386, 388 using the procedure discussed above.

Commercially available straight rongeurs suitable for use in the present system are available from KMedic° under the product designation Intervertebral Disc Rongeurs KM 47-760 and KM 47-780. Commercially available up-biting rongeurs are available from KMedic® under the product designation KM 55-842. Commercially available Modified Wilde-style rongeurs are available from KMedic® under the product designation KM 47-707, KM 47-708, and KM 47-709. Commercially available curved rongeurs are available from Life Instruments under the product name Ferris Smith Pituitary/Foraminotomy Design. Alternatively, any instrument used for nucleus removal can be adapted for use with this system. These instruments include, but are not limited to, flexible ablation devices (Arthrocare's Coblation® technology featured in their SpineWand® instrument), radiofrequency (Ellman International) articulating shavers (Endius MDS Flex Tip® shaver, Clarus Medical Nucleotome®) or rongeurs (Richard Wolf grasping forceps), steerable lasers, water based systems (Hydrocision SpineJet Hydrosurgery System), heat or vaporization based systems.

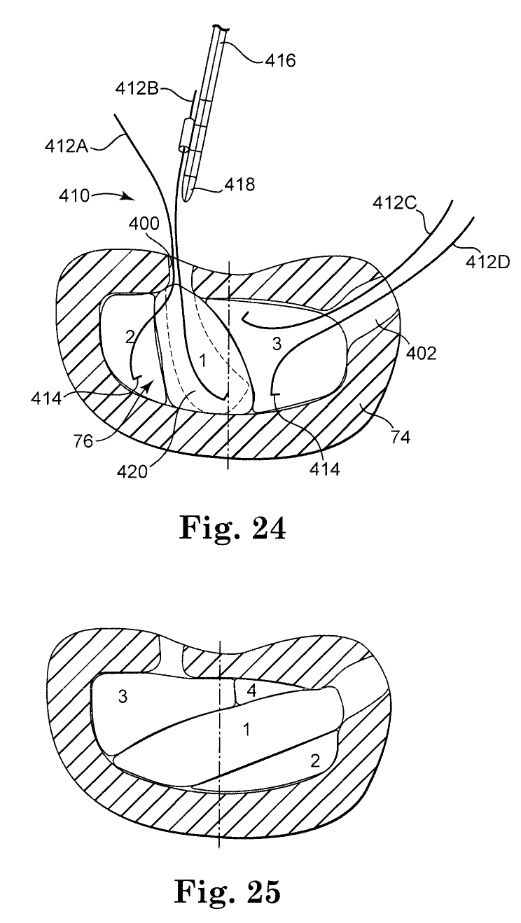

FIG. 24 illustrates an exemplary sequence for performing the nuclectomy using a multi-portal approach. The embodiment of FIG. 24 divides the nucleus into three regions labeled 1, 2, 3. The nuclectomy is preferably performed though one or both of posterior annulotomy 400 and posterolateral annulotomy 402.

The guide system 410 preferably extends into the nucleus 76. In the illustrated embodiment, the guide system 410 includes one or more shaped guide wires 412A, 412B, 412C, 412D (collectively “412”) each preferably with a stop 414. The guide wires 412 are shaped to direct surgical tool 416 to each of the regions 1, 2, 3 within the nucleus 76. More than one guide wire 412 may be required to remove the nucleus material 76 from a single region, such as the guide wires 412C, 412D in region 3. Alternatively, a single guide wire 412 can be repositioned to each of the regions 1, 2, 3 within the nucleus 76.

The guide wires 412 can be rigid or flexible, depending on the application. The guide wires 412 can be used alone or in combination with another guide system, such as the guide system 50 in FIG. 3.

In the illustrated embodiment, the surgical tool 416 slides on a guide wire 412 and has a cutter 418 that cuts a path through the nucleus 76 established by the guide wire 412. The stop 414 limits the travel of the cutter 418. The cutter 418 optionally includes a heated cutting edge. Once the surgical tool 416 reaches the stop 414, the guide wire 412 is repositioned and another section of nucleus material 76 is removed from the annulus 74.

FIG. 25 illustrates an exemplary sequence for performing the nuclectomy using a multi-portal approach. The embodiment of FIG. 25 divides the nucleus into four regions.

In one embodiment, the surgeon performs both sequences so as to maximize removal of nuclear material 76. In another embodiment, the surgeon starts by removing the nucleus material 76 from regions adjacent to the annulotomy 400, and then completes the procedure through the annulotomy 402. Alternatively, the surgeon may switching back and forth between annulotomies 400, 402 until the nucleus is adequately removed. The annulotomies 400, 402 need not have the same number regions, and the number of regions given the approach would depend on the surgeon preference, patient pathology, disc removal from a previous entrance, disc removal instruments, or the type of instrument to be used in the various regions.

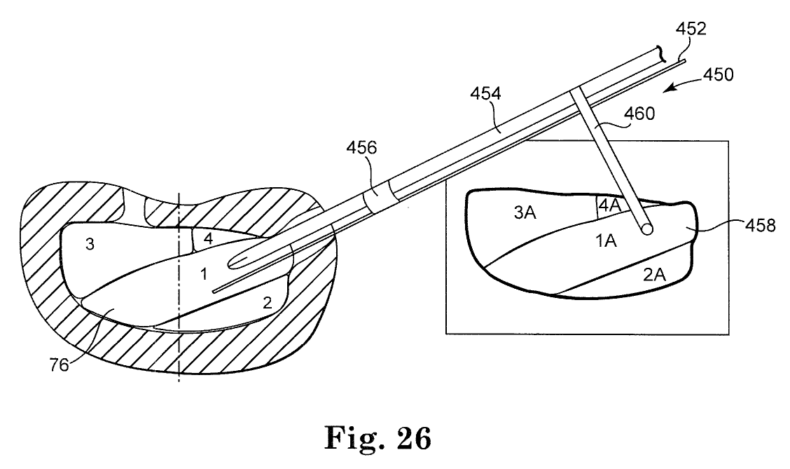

FIG. 26 illustrates an alternate guide system 450 in accordance with an embodiment of the present invention. The guide system 450 includes a tool guide 452 that extends into the nucleus 76 and limits movement of the surgical tool 454. In the illustrated embodiment, the surgical tool 454 is coupled to the tool guide 452 at one or more locations 456.

Template 458 with a shape corresponding generally to the nucleus 76 is coupled to the surgical tool 454 by stylus 460. Movement of the surgical tool 454 within the nucleus 76 is limited by engagement of stylus 458 with template 456. In the illustrated embodiment, the template 458 identifies a plurality of regions 1A, 2A, 3A, 4A, that correspond generally to regions 1, 2, 3, 4 in the nucleus 76. The tool guide 452 is preferably repositioned before performing the nuclectomy in each of the regions 1, 2, 3, 4. The same or a different surgical tool 454 may be used for each of the regions 1, 2, 3, 4. The method and apparatus of FIG. 26 can be used with a single or multiple annulotomies.

Patents and patent applications disclosed herein, including those cited in the Background of the Invention, are hereby incorporated by reference. Other embodiments of the invention are possible. It is to be understood that the above description is intended to be illustrative, and not restrictive. For example, the various guide systems disclosed herein can be combined with any of the adaptors and surgical tools. The surgeon may use a variety of secondary holding devices during a single nuclectomy procedure. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A method for removing at least a portion of a nucleus from an intervertebral disc to prepare a nuclear cavity in an intervertebral disc space to receive an intervertebral prosthesis, the method comprising the steps of:
identifying a plurality of regions in at least a portion of the intervertebral disc;
identifying a sequence for removing the plurality of the regions;
forming at least one annulotomy in an annulus along an annular axis to provide access to the intervertebral disc;
positioning a guide system relative to the at least one annulotomy;
configuring a guide system to limit motion of at least one surgical tool relative to the guide system;
removing a portion of the nucleus from a first region using the surgical tool;
configuring at least one of the guide system and the surgical tool to remove a portion of the nucleus from a second region; and
removing a portion of the nucleus from a second region using the surgical tool.

2. The method of claim 1 comprising the step of positioning the guide system inside the intervertebral disc.

3. The method of claim 1 comprising the step of positioning a distal end of the guide system opposite the annulotomy.

4. The method of claim 1 comprising the steps of configuring at least one of the guide system and a second surgical tool to remove a portion of the nucleus from a second region.

5. The method of claim 1 comprising the steps of configuring at least one of the guide system and a third surgical tool to remove a portion of the nucleus from a third region.

6. The method of claim 1 comprising repeating one or more of the removing steps until at least 70% of the nucleus is removed from the annulus.

7. The method of claim 1 comprising repeating one or more of the removing steps until at least 80% of the nucleus is removed from the annulus.

8. The method of claim 1 comprising repeating one or more of the removing steps until at least 90% of the nucleus is removed from the annulus.

9. The method of claim 1 comprising repeating one or more of the removing steps until the nuclear cavity is generally centered within the intervertebral disc space.

10. The method of claim 1 comprising repeating one or more of the removing steps until the nuclear cavity is symmetrical relative to a midline of a spine.

11. The method of claim 1 comprising the step of forming the annulotomy at a location selected from the posterior, the posterolateral, the lateral, the anterolateral, and the anterior side of the annulus.

12. The method of claim 1 comprising the steps of configuring at least one of the guide system and the surgical tool to limit motion of the surgical tool relative to the guide system to one degree of freedom.

13. The method of claim 1 comprising the steps of configuring at least one of the guide system and the surgical tool to limit motion of the surgical tool relative to the guide system to two degrees of freedom.

14. The method of claim 1 comprising the steps of configuring at least one of the guide system and the surgical tool to linear motion relative to the guide system along a first portion of travel and at least some angular motion along a second portion of travel.

15. The method of claim 1 comprising the step of attaching at least a portion of the guide system to the surgical tool.

16. The method of claim 1 comprising the steps of:
evaluating a geometry of the nucleus;
configuring at least one of the guide system and a plurality of surgical tools to sequentially remove the nucleus; and
performing the nuclectomy method.

17. The method of claim 1 comprising the step of configuring at least one of the guide system and the surgical tool to limit maximum and minimum motion of the surgical tool relative to the guide system.

18. The method of claim 1 comprising the step of providing one or more of visual, auditory or tactile signals to the surgeon in response to motion of the surgical tool relative to the guide system.

19. The method of claim 1 comprising the step of attaching the guide structure to one of a fixed structure or a patient.

20. The method of claim 1 comprising the steps of:
positioning an evaluation mold in the nuclear cavity;
delivering a fluid to the evaluation mold so that the mold substantially fills the nuclear cavity;
estimating the quantity of the nucleus removed from the intervertebral disc space based on the quantity of fluid; and
optionally repeating one or more of the removing steps as necessary until an adequate amount of the nucleus is removed from the intervertebral disc space.

21. The method of claim 1 comprising the steps of:
imaging the intervertebral disc space to estimate a volume of the nucleus; and
comparing the amount of nucleus removed with the estimated volume of the nucleus.

22. The method of claim 1 comprising the steps of:
forming a first annulotomy in the annulus along a first annular axis to provide access to the nucleus;
forming a second annulotomy in the annulus along a second annular axis to provide access to the nucleus;
removing a portion of the nucleus through the first annulotomy; and
removing a portion of the nucleus through the second annulotomy.

23. A method for removing at least a portion of a nucleus from an annulus of an intervertebral disc to prepare a nuclear cavity in an intervertebral disc space to receive an intervertebral prosthesis, the method comprising the steps of:
forming at least one annulotomy in the annulus along an annular axis to provide access to the intervertebral disc;
positioning a guide system relative to the at least one annulotomy;
configuring a guide system to limit motion of at least one surgical tool to linear motion relative to the guide system;
removing a portion of the nucleus from a first region of the intervertebral disc using the surgical tool;
configuring a guide system to limit motion of at least one surgical tool to angular motion relative to the guide system; and
removing a portion of the nucleus from a second region using the surgical tool.

24. A method for removing at least a portion of a nucleus from an annulus of an intervertebral disc to prepare a nuclear cavity in an intervertebral disc space to receive an intervertebral prosthesis, the method comprising the steps of:
forming at least one annulotomy in the annulus along an annular axis to provide access to the intervertebral disc;
positioning a guide system relative to the at least one annulotomy;
configuring a guide system to direct at least one surgical tool to a first region of the nucleus;
removing a portion of the nucleus from the first region using the surgical tool;
configuring a guide system to direct at least one surgical tool to a second region of the nucleus; and
removing a portion of the nucleus from the second region using the surgical tool.
